(19) United States
(12) Patent Application Publication
(10) Pub. No.: US 2022/0264932 A1
(43) Pub. Date: Aug. 25, 2022

(54) AEROSOL PRECURSOR FORMULATIONS

(71) Applicant: RAI Strategic Holdings, Inc., Winston-Salem, NC (US)

(72) Inventors: Karen V. Taluskie, Winston-Salem, NC (US); James N. Figlar, Clemmons, NC (US); Michael F. Davis, Clemmons, NC (US); Kathryn Wilberding, High Point, NC (US); Carolyn Garnett, St. Johns (GB)

(21) Appl. No.: 17/675,620

(22) Filed: Feb. 18, 2022

Related U.S. Application Data
(60) Provisional application No. 63/153,255, filed on Feb. 24, 2021.

(51) Int. Cl. A24B 15/167; A24F 40/10; A24F 42/20; A61M 15/06; A24B 15/32; A24B 15/30

(52) U.S. Cl. CPC A24B 15/167; A24F 40/10; A24F 42/20; A61M 15/06; A24B 15/32; A24B 15/301

(57) ABSTRACT

The disclosure provides liquid aerosol precursor compositions adapted for use in an aerosol delivery device, comprising two or more organic acids. For example, one such composition includes: at least one aerosol former, nicotine, benzoic acid, lactic acid, and levulinic acid, wherein benzoic acid is present in a molar ratio of benzoic acid to nicotine of at least 0.15, wherein lactic acid is present in a molar ratio of lactic acid to nicotine of at least 0.2, and wherein levulinic acid is present in a molar ratio of levulinic acid to nicotine of at least 0.12. The disclosure further provides devices and kits incorporating such compositions.

### CROSS-REFERENCE TO RELATED APPLICATIONS

The present application claims priority and the benefit of U.S. Provisional Patent Application No. 63/153,255, filed Feb. 24, 2021, the disclosure of which is incorporated herein by reference in its entirety.

### TECHNOLOGICAL FIELD

The present disclosure relates to aerosol provision systems such as smoking articles designed to deliver at least one substance to a user, and to formulations for use therein.

### BACKGROUND

Many aerosol provision systems and in particular non-combustible aerosol provision systems have been proposed through the years as improvements upon, or alternatives to, smoking products that require combusting tobacco for use. These systems are generally designed to deliver at least one substance to a user, such as to satisfy a particular “consumer moment.” To this end, the substance may include constituents that impart a physiological effect on the user, a sensorial effect on the user, or both. The substance may be generally present in an aerosol-generating material that may contain one or more constituents of a range of constituents, such as active substances, flavors, aerosol-former materials and other functional materials like fillers.

Aerosol provision systems include, for example, vapor products commonly known as “electronic cigarettes,” “e-cigarettes” or electronic nicotine delivery systems (ENDS), as well as heat-not-burn products including tobacco heating products (THPs) and carbon-tipped tobacco heating products (CTHPs). Many of these products take the form of a system including a device and a consumable, and it is the consumable that includes the material from which the substance to be delivered originates. Typically, the device is reusable, and the consumable is single-use (although some consumables are refillable). Therefore, in many cases, the consumable is sold separately from the device, and often in a multipack. Moreover, subsystems and some individual components of devices or consumables may be sourced from specialist manufacturers.

There remains a need in the art for aerosol provision systems with enhanced sensory characteristics.

### BRIEF SUMMARY

Example implementations of the present disclosure are directed to formulations (liquid aerosol precursor compositions) for use in an aerosol delivery device. Such formulations advantageously comprise, in addition to nicotine and at least one aerosol former, multiple acids (including, e.g., two or more organic acids or three or more organic acids). The use of multiple organic acids, as demonstrated herein, may lead to positive sensory characteristics associated with use of an aerosol delivery device containing such a formulation.

The present disclosure includes, without limitation, the following embodiments.

Embodiment 1: A liquid aerosol precursor composition adapted for use in an aerosol delivery device, comprising: at least one aerosol former, nicotine, benzoic acid, lactic acid, and levulinic acid, wherein benzoic acid is present in a molar ratio of benzoic acid to nicotine of at least 0.15, wherein lactic acid is present in a molar ratio of lactic acid to nicotine of at least 0.2, and wherein levulinic acid is present in a molar ratio of levulinic acid to nicotine of at least 0.12.

Embodiment 2: The liquid aerosol precursor composition of Embodiment 1, wherein the molar ratio of benzoic acid to nicotine is no more than 0.5.

Embodiment 3: The liquid aerosol precursor composition of Embodiment 1 or 2, wherein the molar ratio of lactic acid to nicotine is no more than 0.5.

Embodiment 4: The liquid aerosol precursor composition of any of Embodiments 1 to 3, wherein the molar ratio of levulinic acid to nicotine is no more than 0.6.

Embodiment 5: The liquid aerosol precursor composition of any of Embodiments 1 to 4, wherein the molar ratio of benzoic acid to nicotine is 0.18 to 0.5.

Embodiment 6: The liquid aerosol precursor composition of any of Embodiments 1 to 5, wherein the molar ratio of lactic acid to nicotine is 0.23 to 0.5.

Embodiment 7: The liquid aerosol precursor composition of any of Embodiments 1 to 6, wherein the molar ratio of levulinic acid to nicotine is 0.15 to 0.55.

Embodiment 8: The liquid aerosol precursor composition of any of Embodiments 1 to 7, comprising less than 0.3 molar equivalents of lactic acid to nicotine and less than 0.3 molar equivalents of benzoic acid to nicotine.

Embodiment 9: The liquid aerosol precursor composition of any of Embodiments 1 to 8, wherein the molar ratio of levulinic acid to nicotine is higher than the molar ratio of lactic acid to nicotine and/or higher than the molar ratio of benzoic acid to nicotine.

Embodiment 10: The liquid aerosol precursor composition of any of Embodiments 1 to 9, wherein the molar ratio of levulinic acid to nicotine is higher than the molar ratio of lactic acid to nicotine and higher than the molar ratio of benzoic acid to nicotine.

Embodiment 11: The liquid aerosol precursor composition of any of Embodiments 1 to 10, wherein the combined molar ratio of acids to nicotine is at least 0.7.

Embodiment 12: The liquid aerosol precursor composition of any of Embodiments 1 to 11, wherein nicotine is present in an amount of about 0.5 to about 10% by weight, based on the total weight of the liquid aerosol precursor composition.

Embodiment 13: The liquid aerosol precursor composition of any of Embodiments 1 to 12, wherein the aerosol former comprises at least one polyhydric alcohol.

Embodiment 14: The liquid aerosol precursor composition of any of Embodiments 1 to 13, wherein the aerosol former comprises at least one polyhydric alcohol and the at least one polyhydric alcohol is selected from the group consisting of glycerin, propylene glycol, and mixtures thereof.

Embodiment 15: The liquid aerosol precursor composition of any of Embodiments 1 to 14, wherein the aerosol former comprises one or more polyhydric alcohols, and wherein the one or more polyhydric alcohols are present in an amount of about 50% by weight or higher, based on the total weight of the liquid aerosol precursor composition.

Embodiment 16: The liquid aerosol precursor composition of any of Embodiments 1 to 15, wherein the aerosol former comprises glycerin and propylene glycol, and wherein the glycerin is present in an amount of about 40% by weight or higher and the propylene glycol is present in an amount of about 5% by weight or higher, based on the total weight of the liquid aerosol precursor composition.

Embodiment 17: The liquid aerosol precursor composition of any of Embodiments 1 to 16, wherein the aerosol former comprises glycerin and propylene glycol, and wherein the glycerin is present in an amount of about 40% to about 70% by weight or about 45% to about 70% by weight and the propylene glycol is present in an amount of about 5% to about 40% or about 6% to about 40% (e.g., about 6% to about 26%) by weight, based on the total weight of the liquid aerosol precursor composition.

Embodiment 18: The liquid aerosol precursor composition of any of Embodiments 1 to 17, wherein the liquid aerosol precursor composition comprises no more than about 5% by weight of water, based on the total weight of the liquid aerosol precursor composition.

Embodiment 19: The liquid aerosol precursor composition of any of Embodiments 1 to 18, wherein the liquid aerosol precursor composition comprises no more than about 3% by weight of water, based on the total weight of the liquid aerosol precursor composition.

Embodiment 20: The liquid aerosol precursor composition of any of Embodiments 1 to 15, wherein the liquid aerosol precursor composition is substantially free or completely free of propylene glycol.

Embodiment 21: The liquid aerosol precursor composition of Embodiment 20, wherein the aerosol former comprises glycerin, and wherein the glycerin is present in an amount of at least about 50% by weight (e.g., about 70% to about 90% by weight), based on the total weight of the liquid aerosol precursor composition.

Embodiment 22: The liquid aerosol precursor composition of Embodiment 20 or 21, further comprising about 5% to about 20% water by weight, based on the total weight of the liquid aerosol precursor composition.

Embodiment 23: The liquid aerosol precursor composition of any of Embodiments 1 to 15, wherein the liquid aerosol precursor composition comprises at least about 60% water by weight (e.g., at least about 70% or at least about 75% by weight), based on the total weight of the liquid aerosol precursor composition.

Embodiment 24: The liquid aerosol precursor composition of Embodiment 23, further comprising about 30% glycerin or less by weight (e.g., about 5% to about 30% about 5% to about 20%, or about 5% to about 15% by weight), based on the total weight of the liquid aerosol precursor composition.

Embodiment 25: The liquid aerosol precursor composition of any of Embodiments 1 to 24, wherein the liquid aerosol precursor composition is substantially free or completely free of one or more of phosphoric acid, acetic acid, and pyruvic acid.

Embodiment 26: The liquid aerosol precursor composition of any of Embodiments 1 to 25, wherein the liquid aerosol precursor composition further comprises one or more flavorants in a total amount of about 30% by weight or less, based on the total weight of the liquid aerosol precursor composition.

Embodiment 27: The liquid aerosol precursor composition of any of Embodiments 1 to 26, wherein the liquid aerosol precursor composition has a pH of about 5 to about 7.5.

Embodiment 28: An aerosol delivery device, comprising: a housing enclosing a chamber containing the liquid aerosol precursor composition of any of Embodiments 1 to 27; a heat source in fluid communication with the chamber and configured to heat the liquid aerosol precursor composition to form an aerosol; and an aerosol pathway positioned to carry the aerosol to a mouth-end of the aerosol delivery device.

Embodiment 29: The aerosol delivery device of Embodiment 28, wherein the heat source comprises an electrically powered heating element, and the aerosol delivery device further comprises a power source electronically connected to the heating element.

Embodiment 30: The aerosol delivery device of Embodiment 28 or 29, wherein the aerosol delivery device further comprises a controller configured to control the power transmitted by the power source to the heating element.

Embodiment 31: A kit, comprising: a control body; and one or more cartridges, each cartridge comprising a housing enclosing a chamber containing the liquid aerosol precursor composition of any of Embodiments 1 to 27.

Embodiment 32: The kit of Embodiment 31, further comprising one or more charging components or one or more batteries.

These and other features, aspects, and advantages of the present disclosure will be apparent from a reading of the following detailed description together with the accompanying figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable, unless the context of the disclosure clearly dictates otherwise.

It will therefore be appreciated that this Brief Summary is provided merely for purposes of summarizing some example implementations so as to provide a basic understanding of some aspects of the disclosure. Accordingly, it will be appreciated that the above described example implementations are merely examples and should not be construed to narrow the scope or spirit of the disclosure in any way. Other example implementations, aspects and advantages will become apparent from the following detailed description taken in conjunction with the accompanying figures which illustrate, by way of example, the principles of some described example implementations.

### BRIEF DESCRIPTION OF THE FIGURES

Having thus described aspects of the disclosure in the foregoing general terms, reference will now be made to the accompanying figures, which are not necessarily drawn to scale, and wherein:

FIG. 1 is a block diagram of an aerosol provision system according to some example implementations of the present disclosure;

FIGS. 2 and 3 illustrate an aerosol provision system in the form of a vapor product, according to some example implementations;

FIG. 4 illustrates a nebulizer that may be used to implement an aerosol generator of an aerosol provision system, according to some example implementations; and

FIGS. 5, 6 and 7 illustrate an aerosol provision system in the form of a tobacco heating product (THP), according to some example implementations.

### DETAILED DESCRIPTION

Some implementations of the present disclosure will now be described more fully hereinafter with reference to the accompanying figures, in which some, but not all implementations of the disclosure are shown. Indeed, various implementations of the disclosure may be embodied in many different forms and should not be construed as limited to the implementations set forth herein; rather, these example implementations are provided so that this disclosure will be thorough and complete, and will fully convey the scope of the disclosure to those skilled in the art. Like reference numerals refer to like elements throughout.

Unless specified otherwise or clear from context, references to first, second or the like should not be construed to imply a particular order. A feature described as being above another feature (unless specified otherwise or clear from context) may instead be below, and vice versa; and similarly, features described as being to the left of another feature else may instead be to the right, and vice versa. Also, while reference may be made herein to quantitative measures, values, geometric relationships or the like, unless otherwise stated, any one or more if not all of these may be absolute or approximate to account for acceptable variations that may occur, such as those due to engineering tolerances or the like. As used in the specification and claims, the singular forms “a,” “an,” and “the,” include plural referents unless the context clearly dictates otherwise.

As used herein, unless specified otherwise or clear from context, the “or” of a set of operands is the “inclusive or” and thereby true if and only if one or more of the operands is true, as opposed to the “exclusive or” which is false when all of the operands are true. Thus, for example, “[A] or [B]” is true if [A] is true, or if [B] is true, or if both [A] and [B] are true. Further, the articles “a” and “an” mean “one or more,” unless specified otherwise or clear from context to be directed to a singular form. Furthermore, it should be understood that unless otherwise specified, the terms “data,” “content,” “digital content,” “information,” and similar terms may be at times used interchangeably.

The disclosure provides, for example, an aerosol precursor composition 124 (also referred to herein as “aerosol-generating material”) adapted for use in an aerosol delivery device, wherein the aerosol precursor composition comprises an organic acid component 136, comprising a combination of two or more or three or more different organic acids. Organic acids particularly may be incorporated into the aerosol precursor to affect the flavor, sensation, or organoleptic properties of medicaments, such as nicotine, that may be combined with (or contained within) the liquid aerosol precursor composition. In certain embodiments, an aerosol precursor composition comprising a combination of organic acids can lead to surprisingly improved flavor characteristics associated with an aerosol produced therefrom, as will be described more thoroughly herein below.

Certain examples of organic acids that can be included within the disclosed aerosol precursor compositions include, but are not limited to, acids such as levulinic acid, succinic acid, lactic acid, pyruvic acid, benzoic acid, fumaric acid, combinations thereof, and the like. Inclusion of an organic acid component 136 in aerosol precursor compositions including nicotine may provide a protonated liquid aerosol precursor composition, including nicotine in salt form.

Additional examples of organic acids that can be included within the disclosed aerosol precursor compositions are substituted benzoic acids. Various substituted benzoic acids are known and can be employed in various embodiments. Substituted benzoic acids can have one or more substituents on the benzene ring of benzoic acid and the substituents can be, e.g., ortho, para, and/or meta substituents. Non-limiting examples of substituents can include, for example, hydroxyl groups, halo groups (e.g., chloro, fluoro, bromo, and iodo groups), alkyl groups (e.g., methyl, ethyl, propyl, etc.), alkoxy groups (e.g., methoxy, ethoxy, propoxy, etc.), Specific examples of substituted benzoic acids include, but are not limited to, 2-hydroxybenzoic acid (salicylic acid), 3-hydroxybenzoic acid, 4-hydroxybenzoic acid, 2-methoxybenzoic acid, 2-ethoxybenzoic acid, 3,5-dimethylbenzoic acid, 2,3-dihydroxybenzoic acid (pyrocatechuic acid), 3,5-dihydroxybenzoic acid (α-resorcylic acid), 2,5-dihydroxybenzoic acid (gentisic acid), 3,4-dihydroxybenzoic acid (protocatechuic acid), 4-hydroxy-3-methoxybenzoic acid (vanillic acid), 3-hydroxy-4-methoxybenzoic acid (isovanillic acid), 3,4,5-trihydroxybenzoic acid (gallic acid), 2-hydroxy-6-methylbenzoic acid (6-methyl salicylic acid), 2,4-dihydroxy-6-methylbenzoic acid (orsellinic acid), 4-hydroxy-3,5-dimethoxybenzoic acid (syringic acid), and [3,4-dihydroxy-5-[(3,4,5-trihydroxybenzoyl)oxy]benzoic acid] (digallic acid). In one embodiment, an analogue of a substituted benzoic acid is employed as an organic acid according to the disclosed formulations, e.g., wherein the phenyl ring is replaced with a naphthalene; one example of such an acid is 1-hydroxy-2-naphthoic acid. In any embodiment noted herein, a substituted benzoic acid, including all acids disclosed above, can be substituted for benzoic acid.

Any combination of organic acids can be used as the organic acid component 136 within the disclosed aerosol precursor compositions. In certain embodiments, an aerosol precursor composition is provided that comprises two or more organic acids or three or more organic acids. In particular embodiments, the aerosol precursor composition comprises levulinic acid and benzoic acid; in particular embodiments, the aerosol precursor composition comprises lactic acid and benzoic acid; and in particular embodiments, the aerosol precursor composition comprises lactic acid and levulinic acid. In certain embodiments, phosphoric acid, acetic acid, and/or pyruvic acid are not intentionally added to the aerosol precursor composition; as such, certain aerosol precursor compositions provided herein can be described as being substantially free or completely free of one or more of phosphoric acid, acetic acid, and pyruvic acid. By “substantially free” is meant that the particular acid has not been intentionally added. For example, certain embodiments can be characterized as having less than 0.001% by weight, or less than 0.0001%, or even 0% by weight of one or more of phosphoric acid, acetic acid, and pyruvic acid.

In some embodiments, an aerosol precursor is provided that comprises two organic acids (e.g., a “di-acid” formulation). Various combinations of the above-referenced acids can be employed in certain embodiments. In some such embodiments, a diacid aerosol precursor is provided that particularly comprises lactic acid and benzoic acid.

In certain embodiments, aerosol precursor composition is provided that comprises three organic acids (e.g., referred to herein as a “tri-acid” formulation). Various combinations of three of the above-referenced acids can be employed in certain embodiments, e.g., levulinic, succinic, and lactic acid; levulinic, succinic, and pyruvic acids, levulinic, succinic, and benzoic acids; levulinic, succinic, and fumaric acid; levulinic, lactic, and pyruvic acids; levulinic, lactic, and benzoic acids; levulinic, lactic, and fumaric acids; levulinic, pyruvic, and benzoic acid; levulinic, pyruvic, and fumaric acid; levulinic, benzoic acid, and fumaric acid; succinic, lactic, and pyruvic acids; succinic, lactic, and benzoic acids; succinic, lactic, and fumaric acids; succinic, pyruvic, and benzoic acids; succinic, pyruvic, and fumaric acids; succinic, benzoic, and fumaric acids; lactic, pyruvic, and benzoic acids; lactic, pyruvic, and fumaric acids; and lactic, benzoic, and fumaric acids. In one particular embodiment, the liquid aerosol precursor comprises benzoic acid, lactic acid, and levulinic acid.

Where the aerosol precursor composition comprises two or more (including three or more) organic acids, the molar ratio of the component organic acids with respect to each other, as well as their molar ratio with respect to the molar ratio of nicotine (where also included within the aerosol precursor composition) can vary. In some embodiments, these molar ratios can be adjusted to modify the taste characteristics associated with a vapor produced from the aerosol precursor composition within an aerosol provision device 102 as described in further detail herein below.

In some embodiments, the disclosed aerosol precursor compositions can advantageously be described, in part, by the molar ratio of the organic acids of the organic acid component 136 to nicotine. Generally, the combined molar ratio of all organic acids to nicotine within the aerosol precursor composition is about 0.5 or greater. In some embodiments, the combined molar ratio of all organic acids to nicotine in the aerosol precursor composition is at least 0.7. In some embodiments, the combined molar ratio of all organic acids to nicotine in the aerosol precursor composition is at least 0.71, at least 0.72, at least 0.73, at least 0.74, at least 0.75, at least 0.76, at least 0.77, at least 0.78, at least 0.79, at least 0.8, at least 0.81, at least 0.82, at least 0.83, at least 0.84, at least 0.85, at least 0.86, at least 0.87, at least 0.88, at least 0.89, at least 0.9, at least 0.91, at least 0.92, at least 0.93, at least 0.94, or at least 0.95. Example ranges of molar ratios of the organic acid component 136 (including all organic acids within the composition) to nicotine within various embodiments of the disclosed aerosol precursor compositions include 0.7 to 1.5, 0.7 to 1.2, 0.7 to 1.0, 0.7 to 0.95, 0.7 to 0.9, 0.7 to 0.85, 0.7 to 0.8, 0.75 to 1.5, 0.75 to 0.1, 0.75 to 1.0, 0.75 to 0.95, 0.75 to 0.90, 0.8 to 1.5, 0.8 to 1.2, 0.8 to 1.0, 0.8 to 0.95, 0.85 to 1.5, 0.85 to 1.2, 0.85 to 1.0, or 0.85 to 0.90.

In some embodiments, the organic acid component 134 is included in an amount such that it is less than equimolar, based on total organic acid content, with nicotine (e.g., about 0.7 molar equivalents, about 0.8 molar equivalents, or about 0.9 molar equivalents with respect to nicotine). In some embodiments, the organic acid component 134 is included in an amount such that it is greater than equimolar, based on total organic acid content, with the nicotine (e.g., about 1.1 molar equivalents, about 1.2 molar equivalents, about 1.3 molar equivalents, or about 1.4 molar equivalents with respect to nicotine). In some embodiments, the organic acid component 134 is roughly equimolar, based on total organic acid content, with the nicotine (i.e., about 1 molar equivalent with respect to nicotine).

In tri-acid formulations, in particular, the organic acids of the organic acid component 136 can be provided in roughly equimolar amounts with respect to one another or one or more of the organic acids can be provided in a lesser or greater amount than the other acid(s). In some embodiments incorporating three or more organic acids, all organic acids are included in roughly equimolar amounts. In some embodiments incorporating three or more organic acids, two organic acids are included in roughly equimolar amounts with respect to one another, and a third organic acid is included in a lesser molar amount. In some embodiments incorporating three or more organic acids, two organic acids are included in roughly equimolar amounts with respect to one another, and a third organic acid is included in a greater molar amount. In some embodiments incorporating three or more organic acids, each organic acid is included in a different molar amount.

In some embodiments, the aerosol precursor composition provided herein comprises benzoic acid, with a molar ratio of benzoic acid:nicotine of at least 0.15, e.g., at least 0.16, at least 0.18, at least 0.20, at least 0.22, at least 0.24, or at least 0.25. In some embodiments, the benzoic acid:nicotine molar ratio is no more than 0.5, with example benzoic acid:nicotine molar ratio ranges of 0.15 to 0.50, 0.16 to 0.50, 0.18 to 0.50, 0.20 to 0.50, 0.25 to 0.50, 0.15 to 0.45, 0.15 to 0.40, 0.15 to 0.35, 0.15 to 0.30, or 0.15 to 0.25.

In some embodiments, the aerosol precursor composition comprises lactic acid, with a molar ratio of lactic acid:nicotine of at least 0.20, e.g., at least 0.22, at least 0.23, at least 0.24, at least 0.25, at least 0.26, at least 0.28, or at least 0.30. In some embodiments, the lactic acid:nicotine molar ratio is no more than 0.5, with example lactic acid:nicotine molar ratio ranges of 0.20 to 0.50, 0.22 to 0.50, 0.23 to 0.50, 0.25 to 0.50, 0.26 to 0.50, 0.28 to 0.50, 0.20 to 0.40, 0.20 to 0.35, or 0.20 to 0.30. In some embodiments, the lactic acid:nicotine molar ratio is about 0.4 to about 0.6, e.g., at least about 0.40, at least about 0.42, at least about 0.44, at least about 0.46, or at least about 0.48, e.g., about 0.45 to about 0.55 or 0.48 to about 0.52.

In some embodiments, the aerosol precursor composition comprises levulinic acid, with a molar ratio of levulinic acid:nicotine of at least 0.12, e.g., at least 0.14, at least 0.15, at least 0.16, at least 0.18, at least 0.20, at least 0.22, at least 0.24, at least 0.25, at least 0.26, at least 0.28, or at least 0.30. In some embodiments, the lactic acid:nicotine molar ratio is no more than 0.6, with example lactic acid:nicotine molar ratio ranges of 0.12 to 0.60, 0.14 to 0.60, 0.15 to 0.60, 0.16 to 0.60, 0.18 to 0.60, 0.20 to 0.60, 0.12 to 0.55, 0.14 to 0.55, 0.15 to 0.55, 0.16 to 0.55, 0.18 to 0.55, 0.20 to 0.55, 0.12 to 0.50, 0.14 to 0.50, 0.15 to 0.50 0.16 to 0.50, 0.18 to 0.50, 0.20 to 0.50, 0.20 to 0.55, 0.20 to 0.50, 0.25 to 0.60, or 0.25 to 0.50.

In one particular embodiment, an aerosol precursor composition is provided comprising two organic acids (e.g., a “di-acid” formulation), including lactic acid and benzoic acid. The ratio of the two organic acids with respect to one another can vary, with roughly equivalent molar ratios of each or a greater amount of one or the other. In some such embodiments, it is advantageous to provide the lactic acid in an amount such that the molar ratio of lactic acid is equal to or greater than the molar equivalents of benzoic acid. In some embodiments, it is advantageous to provide the lactic acid in an amount such that the molar equivalent of lactic acid is less than the molar equivalent of benzoic acid. In some embodiments, each of the lactic acid and benzoic acid is present in a molar ratio of acid:nicotine of at least about 0.4, at least about 0.45, or at least about 0.5.

In some embodiments, benzoic acid is included in about 0.4 molar equivalents or greater with respect to nicotine and lactic acid is included in about 0.5 molar equivalents or greater with respect to nicotine. Certain example di-acid formulations include a formulation with a molar ratio of lactic acid to nicotine of about 0.4 and a molar ratio of benzoic acid to nicotine of about 0.46; a formulation with a molar ratio of lactic acid to nicotine of about 0.5 and a molar ratio of benzoic acid to nicotine of about 0.5; and a formulation with a molar ratio of lactic acid to nicotine of about 0.54 and a molar ratio of benzoic acid to nicotine of about 0.46.

In one particular embodiment, an aerosol precursor composition is provided comprising three organic acids, including benzoic acid with a molar ratio of benzoic acid to nicotine of at least 0.15; lactic acid with a molar ratio of lactic acid to nicotine of at least 0.2; and levulinic acid with a molar ratio of levulinic acid to nicotine of at least 0.12, with such molar ratios up to a concentration wherein the total amount of organic acid present is equimolar to the total amount of nicotine present in the aerosol precursor composition. In certain embodiments, one or both of lactic acid and benzoic acid have a molar ratio of acid to nicotine of less than 0.30, such as 0.15 to 0.28 or 0.15 to 0.25.

In certain embodiments, an aerosol precursor composition is provided that comprises three acids, including benzoic acid with a molar ratio of benzoic acid to nicotine of about 0.21; lactic acid with a molar ratio of lactic acid to nicotine of about 0.29; and levulinic acid with a molar ratio of levulinic acid to nicotine of about 0.50. In certain embodiments, an aerosol precursor composition is provided that comprises benzoic acid with a molar ratio of benzoic acid to nicotine of about 0.46; lactic acid with a molar ratio of lactic acid to nicotine of about 0.26; and levulinic acid with a molar ratio of levulinic acid to nicotine of about 0.18.

In some embodiments, it may be advantageous to include levulinic acid in a higher molar ratio than lactic acid or to include levulinic acid in a higher molar ratio than benzoic acid. In certain embodiments, levulinic acid is included in a higher molar ratio than each of lactic acid and benzoic acid. In some embodiments, benzoic acid is included in a higher molar ratio than each of lactic acid and benzoic acid.

The aerosol precursor composition 124 generally comprises, in addition to the organic acid component 136 described above (comprising two or more organic acids), one or more of each of a number of constituents such as an active substance 126, flavorant 128, aerosol-former material 130 or other functional material 132.

The active substance 126 may be a physiologically active material, which is a material intended to achieve or enhance a physiological response such as improved alertness, improved focus, increased energy, increased stamina, increased calm or improved sleep. The active substance may for example be selected from nutraceuticals, nootropics, psychoactives. The active substance may be naturally occurring or synthetically obtained. The active substance may include, for example, nicotine, caffeine, GABA (γ-aminobutyric acid), L-theanine, taurine, theine, vitamins such as B6 or B12 (cobalamin) or C, melatonin, cannabinoids, terpenes, or constituents, derivatives, or combinations thereof. The active substance may include one or more constituents, derivatives or extracts of tobacco, cannabis or another botanical.

In certain embodiments, the active substance 126 comprises nicotine. The amount of nicotine included in such aerosol precursor compositions can vary and, in certain embodiments, is about 0.5 to about 10% by weight, based on the total weight of the aerosol precursor composition. In some embodiments, the amount of nicotine can be about 1 to about 6% by weight, e.g., about 1.5%, about 2%, about 2.5%, about 3%, about 3.5%, about 4%, about 4.5%, or about 5% nicotine by weight, based on the total weight of the aerosol precursor composition.

In some examples in which the active substance 126 includes derivatives or extracts, the active substance may be or include one or more cannabinoids or terpenes.

As noted herein, the active substance 126 may include or be derived from one or more botanicals or constituents, derivatives or extracts thereof. As used herein, the term “botanical” includes any material derived from plants including, but not limited to, extracts, leaves, bark, fibers, stems, roots, seeds, flowers, fruits, pollen, husk, shells or the like. Alternatively, the material may include an active compound naturally existing in a botanical, obtained synthetically. The material may be in the form of liquid, gas, solid, powder, dust, crushed particles, granules, pellets, shreds, strips, sheets, or the like. Example botanicals are tobacco, eucalyptus, star anise, hemp, cocoa, cannabis, fennel, lemongrass, peppermint, spearmint, rooibos, chamomile, flax, ginger, Ginkgo biloba, hazel, hibiscus, laurel, licorice (liquorice), matcha, mate, orange skin, papaya, rose, sage, tea such as green tea or black tea, thyme, clove, cinnamon, coffee, aniseed (anise), basil, bay leaves, cardamom, coriander, cumin, nutmeg, oregano, paprika, rosemary, saffron, lavender, lemon peel, mint, juniper, elderflower, vanilla, wintergreen, beefsteak plant, curcuma, turmeric, sandalwood, cilantro, bergamot, orange blossom, myrtle, cassis, valerian, pimento, mace, damien, marjoram, olive, lemon balm, lemon basil, chive, carvi, verbena, tarragon, geranium, mulberry, ginseng, theanine, theacrine, maca, ashwagandha, damiana, guarana, chlorophyll, baobab or any combination thereof. The mint may be chosen from the following mint varieties: Mentha Arventis, Mentha c.v., Mentha niliaca, Mentha piperita, Mentha piperita citrata c.v., Mentha piperita c.v., Mentha spicata crispa, Mentha cardifolia, Mentha longifolia, Mentha suaveolens variegata, Mentha pulegium, Mentha spicata c.v., and Mentha suaveolens.

In yet other examples, the active substance 126 may be or include one or more of 5-hydroxytryptophan (5-HTP)/oxitriptan/Griffonia simplicifolia, acetylcholine, arachidonic acid (AA, omega-6), ashwagandha (Withania somnifera), Bacopa monniera, beta alanine, beta-hydroxy-beta-methylbutyrate (HMB), Centella asiatica, chai-hu, cinnamon, citicoline, cotinine, creatine, curcumin, docosahexaenoic acid (DHA, omega-3), dopamine, Dorstenia arifolia, Dorstenia Odorata, essential oils, GABA, Galphimia glauca, glutamic acid, hops, kaempferia parviflora (Thai ginseng), kava, L-carnitine, L-arginine, lavender oil, L-choline, liquorice, L-lysine, L-theanine, L-tryptophan, lutein, magnesium, magnesium L-threonate, myo-inositol, nardostachys chinensis, nitrate, oil-based extract of Viola odorata, oxygen, phenylalanine, phosphatidylserine, quercetin, resveratrol, Rhizoma gastrodiae, Rhodiola, Rhodiola rosea, rose essential oil, S-adenosylmethionine (SAMe), sceletium tortuosum, schisandra, selenium, serotonin, skullcap, spearmint extract, spikenard, theobromine, tumaric, Turnera aphrodisiaca, tyrosine, vitamin A, vitamin B3, or yerba mate.

In some example implementations, the aerosol-generating material 124 includes a flavorant 128. As used herein, the terms “flavorant” and “flavor” refer to materials which, where local regulations permit, may be used to create a desired taste, aroma or other somatosensorial sensation in a product for adult consumers. Flavorants may include naturally occurring flavor materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, redberry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. Flavorants may be imitation, synthetic or natural ingredients or blends thereof. Flavorants may be in any suitable form, for example, liquid such as an oil, solid such as a powder, or gas.

In some example implementations, the flavorant 128 may include a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to, eucolyptol or WS-3.

The flavorant 128 can be included within the aerosol precursor composition in varying amounts, which can depend, for example, on the desired sensory characteristics to be associated with use of the aerosol precursor composition and the physical characteristics of the flavorant(s) (including, but not limited to, the volatility of the flavorant and the physical form of the flavorant). In some embodiments, the flavorant 128 (including a single flavorant or a combination of two or more flavorants) is included within the aerosol precursor composition in a total amount of about 40% by weight or less, about 35% by weight or less, about 30% by weight or less, about 25% by weight or less, about 20% by weight or less, about 15% by weight or less, about 10% by weight or less, or about 5% by weight or less, based on the total weight of the aerosol precursor composition. Example ranges of flavorant content are about 2% by weight to about 40% by weight, about 2% by weight to about 30% by weight, about 2% by weight to about 20% by weight, about 2% by weight to about 10% by weight, about 5% by weight to about 40% by weight, about 5% by weight to about 30% by weight, about 5% by weight to about 20% by weight, about 5% by weight to about 10% by weight, about 10% by weight to about 40% by weight, about 10% by weight to about 30% by weight, about 10% by weight to about 20% by weight, about 15% by weight to about 40% by weight, or about 15% by weight to about 30% by weight, based on the total weight of the aerosol precursor composition.

In some embodiments, these values are reported on the basis of “flavor packages,” i.e., flavors dissolved in a carrier liquid (which is a non-limiting manner by which the flavorants disclosed herein are incorporated within the final formulation). The carrier liquid can comprise, e.g., one or more aerosol-former materials. Typically, the amount of aerosol-former material in such flavor packages is greater on a weight/weight basis than the amount of flavorant. As such, relevant amounts of flavorants considered alone within various formulations (excluding any associated aerosol former material within the flavor package) can be, e.g., up to about 20% by weight, up to about 15% by weight, up to about 10% by weight, or up to about 8% by weight based on the final formulation (e.g., about 0.5% to about 20%, about 0.5% to about 15%, about 0.5% to about 10%, or about 0.5% to about 8% by weight).

The aerosol-former material 130 may include one or more constituents capable of forming an aerosol. In some embodiments, the aerosol-former material comprises at least one polyhydric alcohol. In some example implementations, the aerosol-former material may include one or more of glycerine (glycerin), glycerol, propylene glycol, diethylene glycol, triethylene glycol, tetraethylene glycol, 1,3-butylene glycol, erythritol, meso-erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate. In some embodiments, aerosol-former material 130 comprises at least one polyhydric alcohol. Such polyhydric alcohols include, but are not limited to, glycerin, propylene glycol, and mixtures thereof.

The aerosol-former material 130 can be present in varying amounts. In some embodiments, the aerosol-former material is present in an amount of about 50% by weight or more, about 60% by weight or more, or about 70% by weight or more, based on the total weight of the aerosol precursor composition 136.

In some embodiments, the aerosol-former material comprises glycerin and propylene glycol. These components in combination can be present, for example, in the referenced amounts (e.g., about 50% by weight or higher based on the total weight of the aerosol precursor composition 136), and can be present in various ratios with respect to one another, with either component predominating depending upon the intended application. In some embodiments, the glycerin and propylene glycol are present such that the aerosol-former material 130 comprises a higher glycerin content than propylene glycol content. For example, glycerin can be present in an amount of about 40% by weight or higher (e.g., about 40% to about 70% or about 45% to about 70%) based on the total weight of the aerosol precursor composition 136, and propylene glycol can be present in an amount of about 5% by weight or higher (e.g., about 5% to about 40%, about 5% to about 35%, about 5% to about 30%, about 6% to about 26%, about 6% to about 30%, about 6% to about 35%, or about 6% to about 40%) based on the total weight of the aerosol precursor composition 136. In some such embodiments, the amount of water in the liquid aerosol precursor composition is limited, e.g., such that the aerosol precursor composition comprises no more than about 10% by weight of water, no more than 8% by weight of water, no more than about 6% by weight water, no more than about 5% by weight water, no more than about 4% by weight water, or no more than about 3% by weight water, based on the total weight of the aerosol precursor composition 136.

In some embodiments, the aerosol precursor composition 124 comprises a significant amount of water.

For example, in some embodiments, an aerosol precursor composition 124 is provided wherein the aerosol-former material 130 includes at least about 5%, at least about 6%, at least about 7%, at least about 8%, at least about 9%, or at least about 10% water by weight, e.g., about 5% to about 25% water by weight. Accordingly, for example, in some embodiments, the formulated aerosol precursor composition 124 can comprise about 5% to about 20% by weight of water, e.g., about 8% to about 20% or about 8% to about 15% by weight of water, based on the total weight of the aerosol precursor composition 136. In some such embodiments, the aerosol precursor composition further comprises at least one polyhydric alcohol as referenced above, e.g., in an amount of about 50% or greater, about 60% or greater, or about 70% or greater, such as about 70% to about 90% by weight of the polyhydric alcohol(s), based on the total weight of the aerosol precursor composition 124. In particular embodiments, the at least one polyhydric alcohol in such compositions is a polyhydric alcohol other than propylene glycol, e.g., such that the aerosol precursor composition 124 is substantially free or completely free of propylene glycol. For example, the at least one polyhydric alcohol in such embodiments may comprise glycerin. In some such embodiments, comprising about 5% water or greater and about 50% of a polyhydric alcohol or greater (e.g., glycerin), the acids referenced herein above are incorporated at a total of about 1.5 molar equivalents acid(s) to nicotine.

In further embodiments, an aerosol precursor composition 124 is provided wherein the aerosol-former material 130 includes primarily water, e.g., at least about 70% by weight, at least about 75% by weight, at least about 80% by weight, or at least about 85% by weight, e.g., about 75% to about 95% by weight or about 85% to about 95% by weight. Accordingly, for example, in some embodiments, the formulated aerosol precursor composition 124 can comprise at least about 60% water by weight, at least about 65% water by weight, at least about 70% water by weight, or at least about 75% water by weight, e.g., about 60% to about 85% water by weight, about 70% to about 85% water by weight, or about 75% to about 85% water by weight. In some such embodiments, the aerosol precursor composition further comprises at least one polyhydric alcohol as referenced above, e.g., in an amount of about 30% or less, 25% or less, 20% or less, or about 15% or less, such as about 5% to about 30% or about 5% to about 15% by weight, based on the total weight of the aerosol precursor composition 124. In particular embodiments, the at least one polyhydric alcohol in such compositions is a polyhydric alcohol other than propylene glycol, e.g., such that the aerosol precursor composition 124 is substantially free or completely free of propylene glycol. For example, the at least one polyhydric alcohol in such embodiments may comprise glycerin. In some such embodiments, comprising about 65% water or greater and about 50% of a polyhydric alcohol or greater (e.g., glycerin), the acids referenced herein above are incorporated at a total of about 1 molar equivalent acid(s) to nicotine.

In certain embodiments, the referenced values of water content are indicative of the water intentionally added to the formulation (additional water may be unintentionally incorporated over time, depending, e.g., on the environment and packaging in which the formulations are stored).

The one or more other functional materials 132 may include one or more of pH regulators, colouring agents, preservatives, binders, fillers, stabilizers, and/or antioxidants. Suitable binders include, for example, pectin, guar gum, fruit pectin, citrus pectin, tobacco pectin, hydroxyethyl guar gum, hydroxypropyl guar gum, hydroxyethyl locust bean gum, hydroxypropyl locust bean gum, alginate, starch, modified starch, derivatized starch, methyl cellulose, ethyl cellulose, ethylhydroxymethyl cellulose, carboxymethyl cellulose, tamarind gum, dextran, pullalon, konjac flour or xanthan gum.

The pH of the aerosol precursor composition 124 can vary and can be dependent, e.g., on the content and composition of the organic acid component 134. In some embodiments, the pH of the aerosol precursor composition is about 4 to about 7.5, e.g., about 5 to about 7.5, e.g., about 5.5 to about 7.5.

An aerosol delivery device according to the present disclosure may take on a variety of embodiments, as discussed in detail below. However, typically, the use of the aerosol delivery device by a consumer will be similar in scope. The foregoing description of the aerosol precursor composition is applicable to the various embodiments described through minor modifications, which are apparent to the person of skill in the art in light of the further disclosure provided herein. The description of use, however, is not intended to limit the use of the aerosol precursor composition 124 but is provided to comply with all necessary requirements of disclosure herein.

The aerosol precursor composition 124 described herein can be included within an article of a consumable 104, part or all of which is intended to be consumed during use by a user. An aerosol provision system 100 may include one or more consumables, and each consumable may include one or more liquid aerosol precursor compositions (which can be as described herein or which can be alternative types of compositions). In some examples in which the aerosol provision system is a hybrid product, the aerosol provision system may include a liquid aerosol precursor composition to generate an aerosol, which may then pass through a second, solid aerosol-generating material to pick up additional constituents before reaching the user. These aerosol-generating materials may be within a single consumable or respective consumables that may be separately removable.

The aerosol precursor composition 124 is capable of generating aerosol, for example when heated, irradiated or energized in any other way. The aerosol precursor composition may be, for example, in the form of a solid, semi-solid, liquid or gel. In certain preferred embodiments, the aerosol precursor composition 124 is a liquid aerosol precursor composition. The aerosol precursor composition may include an “amorphous solid,” which may be alternatively referred to as a “monolithic solid” (i.e., non-fibrous). In some examples, the amorphous solid may be a dried gel. The amorphous solid is a solid material that may retain some fluid, such as liquid, within it. In some examples, the aerosol precursor composition may include from about 50 wt %, 60 wt % or 70 wt % of amorphous solid, to about 90 wt %, 95 wt % or 100 wt % of amorphous solid.

Example implementations of the present disclosure are generally directed to delivery systems designed to deliver at least one substance to a user, such as to satisfy a particular “consumer moment.” The substance may include constituents that impart a physiological effect on the user, a sensorial effect on the user, or both.

As referenced above, by employing an organic acid component 136 comprising three different organic acids, the sensory characteristics of the aerosol formed from the aerosol precursor composition within the delivery system may be modified and, in some embodiments, surprising (e.g., positive) sensory/taste characteristics are associated with the inclusion of such an organic acid component.

Delivery systems for the disclosed aerosol precursor composition provided herein may take many forms. Examples of suitable delivery systems include aerosol provision systems such as powered aerosol provision systems designed to release one or more substances or compounds from an aerosol-generating material without combusting the aerosol-generating material. These aerosol provision systems may at times be referred to as non-combustible aerosol provision systems, aerosol delivery devices or the like, and the aerosol-generating material may be, for example, in the form of a solid, semi-solid, liquid or gel and may or may not contain nicotine.

Examples of suitable aerosol provision systems include vapor products, heat-not-burn products, hybrid products and the like. Vapor products are commonly known as “electronic cigarettes,” “e-cigarettes” or electronic nicotine delivery systems (ENDS), although the aerosol-generating material need not include nicotine. Many vapor products are designed to heat a liquid material to generate an aerosol. Other vapor products are designed to break up an aerosol-generating material into an aerosol without heating, or with only secondary heating. Heat-not-burn products include tobacco heating products (THPs) and carbon-tipped tobacco heating products (CTHPs), and many are designed to heat a solid material to generate an aerosol without combusting the material.

Hybrid products use a combination of aerosol-generating materials, one or a plurality of which may be heated. Each of the aerosol-generating materials may be, for example, in the form of a solid, semi-solid, liquid, or gel. Some hybrid products are similar to vapor products except that the aerosol generated from a liquid or gel aerosol-generating material passes through a second material (such as tobacco) to pick up additional constituents before reaching the user. In some example implementations, the hybrid system includes a liquid or gel aerosol-generating material, and a solid aerosol-generating material. A solid aerosol-generating material may include, for example, tobacco or a non-tobacco product.

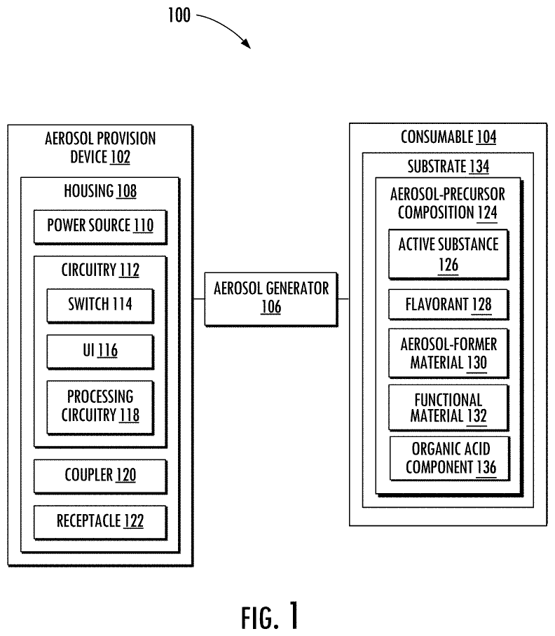

FIG. 1 is a block diagram of an aerosol provision system 100 according to some example implementations, incorporating the aerosol precursor composition provided herein. In various examples, the aerosol provision system may be a vapor product, heat-not-burn product or hybrid product. The aerosol provision system includes one or more of each of a number of components including, for example, an aerosol provision device 102, and a consumable 104 (sometimes referred to as an article) for use with the aerosol provision device. The aerosol provision system also includes an aerosol generator 106. In various implementations, the aerosol generator may be part of the aerosol provision device or the consumable. In other implementations, the aerosol generator may be separate from the aerosol provision device and the consumable, and removably engaged with the aerosol provision device and/or the consumable.

In various examples, the aerosol provision system 100 and its components including the aerosol provision device 102 and the consumable 104 may be reusable or single-use. In some examples, the aerosol provision system including both the aerosol provision device and the consumable may be single use. In some examples, the aerosol provision device may be reusable, and the consumable may be reusable (e.g., refillable) or single use (e.g., replaceable). In yet further examples, the consumable may be both refillable and also replaceable. In examples in which the aerosol generator 106 is part of the aerosol provision device or the consumable, the aerosol generator may be reusable or single-use in the same manner as the aerosol provision device or the consumable.

In some example implementations, the aerosol provision device 102 may include a housing 108 with a power source 110 and circuitry 112. The power source is configured to provide a source of power to the aerosol provision device and thereby the aerosol provision system 100. The power source may be or include, for example, an electric power source such as a non-rechargeable battery or a rechargeable battery, solid-state battery (SSB), lithium-ion battery, supercapacitor, or the like.

The circuitry 112 may be configured to enable one or more functionalities (at times referred to as services) of the aerosol provision device 102 and thereby the aerosol provision system 100. The circuitry includes electronic components, and in some examples one or more of the electronic components may be formed as a circuit board such as a printed circuit board (PCB).

In some examples, the circuitry 112 includes at least one switch 114 that may be directly or indirectly manipulated by a user to activate the aerosol provision device 102 and thereby the aerosol provision system 100. The switch may be or include a pushbutton, touch-sensitive surface or the like that may be operated manually by a user. Additionally or alternatively, the switch may be or include a sensor configured to sense one or more process variables that indicate use of the aerosol provision device or aerosol provision system. One example is a flow sensor, pressure sensor, pressure switch or the like that is configured to detect airflow or a change in pressure caused by airflow when a user draws on the consumable 104.

The switch 114 may provide user interface functionality. In some examples, the circuitry 112 may include a user interface (UI) 116 that is separate from or that is or includes the switch. The UI may include one or more input devices and/or output devices to enable interaction between the user and the aerosol provision device 102. As described above with respect to the switch, examples of suitable input devices include pushbuttons, touch-sensitive surfaces and the like. The one or more output devices generally include devices configured to provide information in a human-perceptible form that may be visual, audible or tactile/haptic. Examples of suitable output devices include light sources such as light-emitting diodes (LEDs), quantum dot-based LEDs and the like. Other examples of suitable output devices include display devices (e.g., electronic visual displays), touchscreens (integrated touch-sensitive surface and display device), loudspeakers, vibration motors and the like.

In some examples, the circuitry 112 includes processing circuitry 118 configured to perform data processing, application execution, or other processing, control or management services according to one or more example implementations. The processing circuitry may include a processor embodied in a variety of forms such as at least one processor core, microprocessor, coprocessor, controller, microcontroller or various other computing or processing devices including one or more integrated circuits such as, for example, an ASIC (application specific integrated circuit), an FPGA (field programmable gate array), some combination thereof, or the like. In some examples, the processing circuitry may include memory coupled to or integrated with the processor, and which may store data, computer program instructions executable by the processor, some combination thereof, or the like.

As also shown, in some examples, the housing 108 and thereby the aerosol provision device 102 may also include a coupler 120 and/or a receptacle 122 structured to engage and hold the consumable 104, and thereby couple the aerosol provision device with the consumable. The coupler may be or include a connector, fastener or the like that is configured to connect with a corresponding coupler of the consumable, such as by a press fit (or interference fit) connection, threaded connection, magnetic connection or the like. The receptacle may be or include a reservoir, tank, container, cavity, receiving chamber or the like that is structured to receive and contain the consumable or at least a portion of the consumable.

In some example implementations, the aerosol-generating material 124 may be present on or in a support to form a substrate 134. The support may be or include, for example, paper, card, paperboard, cardboard, reconstituted material (e.g., a material formed from reconstituted plant material, such as reconstituted tobacco, reconstituted hemp, etc.), a plastics material, a ceramic material, a composite material, glass, a metal, or a metal alloy. In some examples, the support includes a susceptor, which may be embedded within the aerosol-generating material, or on one or either side of the aerosol-generating material.

Although not separately shown, in some example implementations, the consumable 104 may further include receptacle structured to engage and hold the aerosol-generating material 124, or substrate 134 with the aerosol-generating material. The receptacle may be or include a reservoir, tank, container, cavity, receiving chamber or the like that is structured to receive and contain the aerosol-generating material or the substrate. The consumable may include an aerosol-generating material transfer component (also referred to as a liquid transport element) configured to transport aerosol-generating material to the aerosol generator 106. The aerosol-generating material transfer component may be adapted to wick or otherwise transport aerosol-generating material via capillary action. In some examples, the aerosol-generating material transfer component may include a microfluidic chip, a micro pump or other suitable component to transport aerosol-generating material.

The aerosol generator 106 (also referred to as an atomizer, aerosolizer or aerosol production component) is configured to energize the aerosol-generating material 124 to generate an aerosol, or otherwise cause generation of an aerosol from the aerosol-generating material. More particularly, in some examples, the aerosol generator may be powered by the power source 110 under control of the circuitry 112 to energize the aerosol-generating material to generate an aerosol.

In some example implementations, the aerosol generator 106 is an electric heater configured to perform electric heating in which electrical energy from the power source is converted to heat energy, which the aerosol-generating material is subject to so as to release one or more volatiles from the aerosol-generating material to form an aerosol. Examples of suitable forms of electric heating include resistance (Joule) heating, induction heating, dielectric and microwave heating, radiant heating, arc heating and the like. More particular examples of suitable electric heaters include resistive heating elements such as wire coils, flat plates, prongs, micro heaters or the like.

In some example implementations, the aerosol generator 106 is configured to cause an aerosol to be generated from the aerosol-generating material without heating, or with only secondary heating. For example, the aerosol generator may be configured to subject the aerosol-generating material to one or more of increased pressure, vibration, or electrostatic energy. More particular examples of these aerosol generators include jet nebulizers, ultrasonic wave nebulizers, vibrating mesh technology (VMT) nebulizers, surface acoustic wave (SAW) nebulizers, and the like.

A jet nebulizer is configured to use compressed gas (e.g., air, oxygen) to break up aerosol-generating material 124 into an aerosol, and an ultrasonic wave nebulizer is configured to use ultrasonic waves to break up aerosol-generating material into an aerosol. A VMT nebulizer includes a mesh, and a piezo material (e.g., piezoelectric material, piezomagnetic material) that may be driven to vibrate and cause the mesh to break up aerosol-generating material into an aerosol. A SAW nebulizer is configured to use surface acoustic waves or Rayleigh waves to break up aerosol-generating material into an aerosol.

In some examples, the aerosol generator 106 may include a susceptor, or the susceptor may be part of the substrate 134. The susceptor is a material that is heatable by penetration with a varying magnetic field generated by a magnetic field generator that may be separate from or part of the aerosol generator. The susceptor may be an electrically-conductive material, so that penetration thereof with a varying magnetic field causes induction heating of the heating material. The heating material may be magnetic material, so that penetration thereof with a varying magnetic field causes magnetic hysteresis heating of the heating material. The susceptor in some examples may be both electrically-conductive and magnetic, so that the susceptor of these examples is heatable by both heating mechanisms.

Although not separately shown, either or both the aerosol provision device 102 or the consumable 104 may include an aerosol-modifying agent. The aerosol-modifying agent is a substance configured to modify the aerosol generated from the aerosol-generating material 124, such as by changing the taste, flavor, acidity or another characteristic of the aerosol. In various examples, the aerosol-modifying agent may be an additive or a sorbent. The aerosol-modifying agent may include, for example, one or more of a flavorant, colorant, water or carbon adsorbent. The aerosol-modifying agent may be a solid, semi-solid, liquid or gel. The aerosol-modifying agent may be in powder, thread or granule form. The aerosol-modifying agent may be free from filtration material. In some examples, the aerosol-modifying agent may be provided in an aerosol-modifying agent release component, that is operable to selectively release the aerosol-modifying agent.

The aerosol provision system 100 and its components including the aerosol provision device 102, consumable 104, and aerosol generator 106 may be manufactured with any of a number of different form factors, and with additional or alternative components relative to those described above.

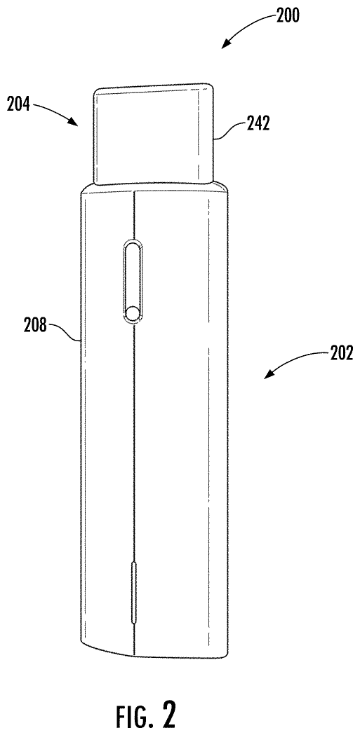

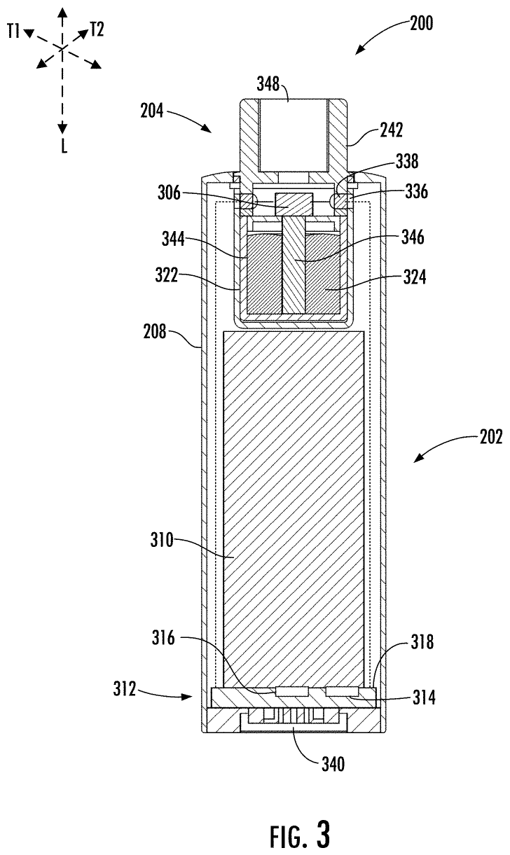

FIGS. 2 and 3 illustrate an aerosol provision system 200 in the form of a vapor product, and that in some example implementations may correspond to the aerosol provision system 100. As shown, the aerosol provision system 200 may include an aerosol provision device 202 (also referred to as a control body or power unit) and a consumable 204 (also referred to as a cartridge or tank), which may correspond to respectively the aerosol provision device 102 and the consumable 104. The aerosol provision system and in particular the consumable may also include an aerosol generator corresponding to the aerosol generator 106, and in the form of an electric heater 306 such as a heating element like a metal wire coil configured to convert electrical energy to heat energy through resistance (Joule) heating. The aerosol provision device and the consumable can be permanently or detachably aligned in a functioning relationship. FIGS. 2 and 3 illustrate respectively a perspective view and a partially cut-away side view of the aerosol provision system in a coupled configuration.

As seen in FIG. 2 and the cut-away view illustrated in FIG. 3, the aerosol provision device 202 and consumable 204 each include a number of respective components. The components illustrated in FIG. 3 are representative of the components that may be present in an aerosol provision device and consumable and are not intended to limit the scope of components that are encompassed by the present disclosure.

The aerosol provision device 202 may include a housing 208 (sometimes referred to as an aerosol provision device shell) that may include a power source 310. The housing may also include circuitry 312 with a switch in the form of a sensor 314, a user interface including a light source 316 that may be illuminated with use of the aerosol provision system 200, and processing circuitry 318 (also referred to as a control component). The housing may also include a receptacle in the form of a consumable receiving chamber 322 structured to engage and hold the consumable 204. And the consumable may include an aerosol precursor composition 324 that may correspond to aerosol precursor composition 124 as described herein and that may include, in addition to the organic acid component, one or more of each of a number of constituents such as an active substance, flavorant, aerosol-former material or other functional material.

As also seen in FIG. 3, the aerosol provision device 202 may also include electrical connectors 336 positioned in the consumable receiving chamber 322 configured to electrically couple the circuitry and thereby the aerosol provision device with the consumable 204, and in particular electrical contacts 338 on the consumable. In this regard, the electrical connectors and electrical contacts may form a connection interface of the aerosol provision device and consumable. As also shown, the aerosol provision device may include an external electrical connector 340 to connect the aerosol provision device with one or more external devices. Examples of suitable external electrical connectors include USB connectors, proprietary connectors such as Apple's Lightning connector, and the like.

In various examples, the consumable 204 includes a tank portion and a mouthpiece portion. The tank portion and the mouthpiece portion may be integrated or permanently fixed together, or the tank portion may itself define the mouthpiece portion (or vice versa). In other examples, the tank portion and the mouthpiece portion may be separate and removably engaged with one another.

The consumable 204, tank portion and/or mouthpiece portion may be separately defined in relation to a longitudinal axis (L), a first transverse axis (T1) that is perpendicular to the longitudinal axis, and a second transverse axis (T2) that is perpendicular to the longitudinal axis and is perpendicular to the first transverse axis. The consumable can be formed of a housing 342 (sometimes referred to as the consumable shell) enclosing a reservoir 344 (in the tank portion) configured to retain the aerosol-generating material 324. In some examples, the consumable may include an aerosol generator, such as electric heater 306 in the illustrated example. In some examples, the electrical connectors 336 on the aerosol provision device 202 and electrical contacts 338 on the consumable may electrically connect the electric heater with the power source 310 and/or circuitry 312 of the aerosol provision device.

As shown, in some examples, the reservoir 344 may be in fluid communication with an aerosol-generating material transfer component 346 adapted to wick or otherwise transport aerosol-generating material 324 stored in the reservoir housing to the electric heater 306. At least a portion of the aerosol-generating material transfer component may be positioned proximate (e.g., directly adjacent, adjacent, in close proximity to, or in relatively close proximity to) the electric heater. The aerosol-generating material transfer component may extend between the electric heater and the aerosol-generating material stored in the reservoir, and at least a portion of the electric heater may be located above a proximal end the reservoir. For the purposes of the present disclosure, it should be understood that the term “above” in this particular context should be interpreted as meaning toward a proximal end of the reservoir and/or the consumable 204 in direction substantially along the longitudinal axis (L). Other arrangements of the aerosol-generating material transfer component are also contemplated within the scope of the disclosure. For example, in some example implementations, the aerosol-generating material transfer component may be positioned proximate a distal end of the reservoir and/or arranged transverse to the longitudinal axis (L).

The electric heater 306 and aerosol-generating material transfer component 346 may be configured as separate elements that are fluidly connected, the electric heater and aerosol-generating material transfer component or may be configured as a combined element. For example, in some implementations an electric heater may be integrated into an aerosol-generating material transfer component. Moreover, the electric heater and the aerosol-generating material transfer component may be formed of any construction as otherwise described herein. In some examples, a valve may be positioned between the reservoir 344 and electric heater, and configured to control an amount of aerosol-generating material 324 passed or delivered from the reservoir to the electric heater.

An opening 348 may be present in the housing 342 (e.g., at the mouth end of the mouthpiece portion) to allow for egress of formed aerosol from the consumable 204.

As indicated above, the circuitry 312 of the aerosol provision device 202 may include a number of electronic components, and in some examples may be formed of a circuit board such as a PCB that supports and electrically connects the electronic components. The sensor 314 (switch) may be one of these electronic components positioned on the circuit board. In some examples, the sensor may comprise its own circuit board or other base element to which it can be attached. In some examples, a flexible circuit board may be utilized. A flexible circuit board may be configured into a variety of shapes. In some examples, a flexible circuit board may be combined with, layered onto, or form part or all of a heater substrate.

In some examples, the reservoir 344 may be a container for storing the aerosol precursor composition 324. In some examples, the reservoir may be or include a fibrous reservoir with a substrate with the aerosol-generating material present on or in a support. For example, the reservoir can comprise one or more layers of nonwoven fibers substantially formed into the shape of a tube encircling the interior of the housing 342, in this example. The aerosol-generating material may be retained in the reservoir. Liquid components, for example, may be sorptively retained by the reservoir. The reservoir may be in fluid connection with the aerosol-generating material transfer component 346. The aerosol-generating material transfer component may transport the aerosol-generating material stored in the reservoir via capillary action— or via a micro pump— to the electric heater 306. As such, the electric heater is in a heating arrangement with the aerosol-generating material transfer component.

In use, when a user draws on the aerosol provision system 200, airflow is detected by the sensor 314, and the electric heater 306 is activated to energize the aerosol-generating material 324 to generate an aerosol. Drawing upon the mouth end of the aerosol provision system causes ambient air to enter and pass through the aerosol provision system. In the consumable 204, the drawn air combines with the aerosol that is whisked, aspirated or otherwise drawn away from the electric heater and out the opening 348 in the mouth end of the aerosol provision system.

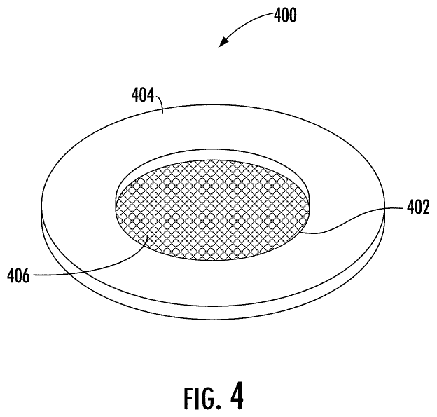

Again, as shown in FIGS. 2 and 3, the aerosol generator of the aerosol provision system 200 is an electric heater 306 designed to heat the aerosol-generating material 324 to generate an aerosol. In other implementations, the aerosol generator is designed to break up the aerosol-generating material without heating, or with only secondary heating. FIG. 4 illustrates a nebulizer 400 that may be used to implement the aerosol generator of an aerosol provision system, according to some these other example implementations.

As shown in FIG. 4, the nebulizer 400 includes a mesh plate 402 and a piezo material 404 that may be affixed to one another. The piezo material may be driven to vibrate and cause the mesh plate to break up aerosol-generating material into an aerosol. In some examples, the nebulizer may also include a supporting component located on a side of the mesh plate opposite the piezo material to increase the longevity of the mesh plate, and/or an auxiliary component between the mesh plate and the piezo material to facilitate interfacial contact between the mesh plate and the piezo material.

In various example implementations, the mesh plate 402 may have a variety of different configurations. The mesh plate may have a flat profile, a domed shape (concave or convex with respect to the aerosol-generating material), or a flat portion and a domed portion. The mesh plate defines a plurality of perforations 406 that may be substantially uniform or vary in size across a perforated portion of the mesh plate. The perforations may be circular openings or non-circular openings (e.g., oval, rectangular, triangular, regular polygon, irregular polygon). In three-dimensions, the perforations may have a fixed cross section such as in the case of cylindrical perforations with a fixed circular cross section, or a variable cross section such as in the case of truncated cone perforations with a variable circular cross section. In other implementations, the perforations may be tetragonal or pyramidal.

The piezo material 404 may be or include a piezoelectric material or a piezomagnetic material. A piezoelectric material may be coupled to circuitry configured to produce an oscillating electric signal to drive the piezoelectric material to vibrate. For a piezomagnetic material, the circuitry may produce a pair of antiphase, oscillating electric signals to drive a pair of magnets to produce antiphase, oscillating magnetic fields that drives the piezomagnetic material to vibrate.

The piezo material 404 may be affixed to the mesh plate 402, and vibration of the piezo material may in turn cause the mesh plate to vibrate. The mesh plate may be in contact with or immersed in aerosol-generating material, in sufficient proximity of aerosol-generating material, or may otherwise receive aerosol-generating material via an aerosol-generating material transfer component. The vibration of the mesh plate, then, may cause the aerosol-generating material to pass through the perforations 406 that break up the aerosol-generating material into an aerosol. More particularly, in some examples, aerosol-generating material may be driven through the perforations 406 in the vibrating mesh plate 402 resulting in aerosol particles. In other examples in which the mesh plate is in contact with or immersed in aerosol-generating material, the vibrating mesh plate may create ultrasonic waves within aerosol-generating material that cause formation of an aerosol at the surface of the aerosol-generating material.

As described above, hybrid products use a combination of aerosol-generating materials, and some hybrid products are similar to vapor products except that the aerosol generated from one aerosol-generating material may pass through a second aerosol-generating material to pick up additional constituents. Another similar aerosol provision system in the form of a hybrid product may therefore be constructed similar to the vapor product in FIGS. 2 and 3 (with an electric heater 306 or a nebulizer 400). The hybrid product may include a second aerosol-generating material through which aerosol from the aerosol-generating material 324 is passed to pick up additional constituents before passing through the opening 348 in the mouth end of the aerosol provision system.

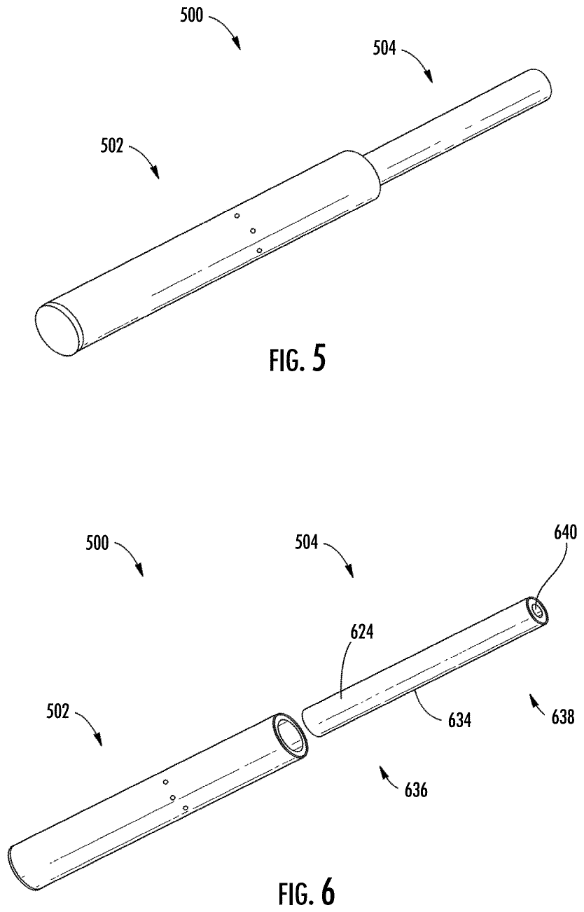

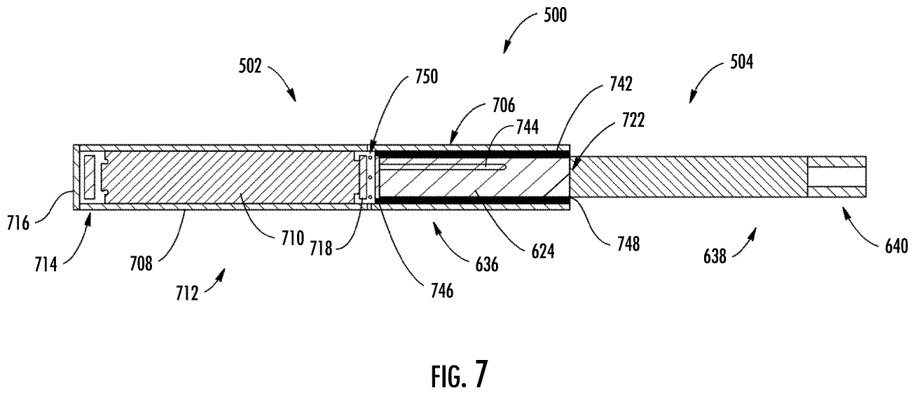

FIGS. 5, 6 and 7 illustrate an aerosol provision system 500 in the form of a heat-not-burn product, and that in some example implementations may correspond to the aerosol provision system 100. As shown, the aerosol provision system may include an aerosol provision device 502 (also referred to as a control body or power unit) and a consumable 504 (also referred to as an aerosol source member), which may correspond to respectively the aerosol provision device 102 and the consumable 104. The aerosol provision system and in particular the aerosol provision device may also include an aerosol generator corresponding to the aerosol generator 106, and in the form of an electric heater 706. The aerosol provision device and the consumable can be permanently or detachably aligned in a functioning relationship. FIG. 5 illustrates the aerosol provision system in a coupled configuration, whereas FIG. 6 illustrates the aerosol provision system in a decoupled configuration. FIG. 7 illustrates a partially cut-away side view of the aerosol provision system in the coupled configuration.

As seen in FIGS. 5, 6 and 7, the aerosol provision device 502 and consumable 504 each include a number of respective components. The components illustrated in the figures are representative of the components that may be present in an aerosol provision device and consumable and are not intended to limit the scope of components that are encompassed by the present disclosure.

The aerosol provision device 502 may include a housing 708 (sometimes referred to as an aerosol provision device shell) that may include a power source 710. The housing may also include circuitry 712 with a switch in the form of a sensor 714, a user interface including a light source 716 that may be illuminated with use of the aerosol provision system 500, and processing circuitry 718 (also referred to as a control component). In some examples, at least some of the electronic components of the circuitry may be formed of a circuit board or a flexible circuit board that supports and electrically connects the electronic components.

The housing 708 may also include a receptacle in the form of a consumable receiving chamber 722 structured to engage and hold the consumable 504. The consumable may include an aerosol precursor composition 624 that may correspond to aerosol precursor composition 124, and that may include one or more of each of a number of constituents in addition to the organic acid component described above, such as an active substance, flavorant, aerosol-former material or other functional material. In some embodiments, the aerosol precursor composition may be present on or in a support to form a substrate 634.

In the coupled configuration of the aerosol provision system 500, the consumable 504 may be held in the receiving chamber 722 in varying degrees. In some examples, less than half or approximately half of the consumable may be held in the receiving chamber. In other examples, more than half of the consumable may be held in the receiving chamber. In yet other examples, substantially the entire consumable may be held in the receiving chamber.

As shown in FIGS. 6 and 7, in various implementations of the present disclosure, the consumable 504 may include a heated end 636 sized and shaped for insertion into the aerosol provision device 502, and a mouth end 638 upon which a user draws to create the aerosol. In various implementations, at least a portion of the heated end may include the aerosol-generating material 624.

In some example implementations, the mouth end 608 of the consumable 504 may include a filter 640 made of a material such as cellulose acetate or polypropylene. The filter may additionally or alternatively contain strands of tobacco containing material. In some examples, at least a portion of the consumable may be wrapped in an exterior overwrap material, which may be formed of any material useful to provide additional structure, support and/or thermal resistance. In some examples, an excess length of the overwrap at the mouth end of the consumable may function to simply separate the aerosol-generating material 624 from the mouth of a user or to provide space for positioning of a filter material, or to affect draw on the consumable or to affect flow characteristics of the aerosol leaving the consumable during draw.

The electric heater 706 may perform electric heating of the aerosol-generating material 624 by resistance (Joule) heating, induction heating, dielectric and microwave heating, radiant heating, arc heating and the like. The electric heater may have a variety of different configurations. In some examples, at least a portion of the electric heater may surround or at least partially surround at least a portion of the consumable 504 including the aerosol-generating material when inserted in the aerosol provision device 502. In other examples, at least a portion of the electric heater may penetrate the consumable when the consumable is inserted into the aerosol provision device. In some examples, the substrate 634 material may include a susceptor, which may be embedded within the aerosol-generating material, or on one or either side of the aerosol-generating material.

Although shown as a part of the aerosol provision device 502, the electric heater 706 may instead be a part of the consumable 504. In some examples, the electric heater or a part of the electric heater may be may be combined, packaged or integral with (e.g., embedded within) the aerosol-generating material 624.

As shown, in some examples, the electric heater 706 may extend proximate an engagement end of the housing 708, and may be configured to substantially surround a portion of the heated end 636 of the consumable 504 that includes the aerosol-generating material 624. The electric heater 706 may be or may include an outer cylinder 742, and one or more resistive heating elements 744 such as prongs surrounded by the outer cylinder to create the receiving chamber 722, which may extend from a receiving base 746 of the aerosol provision device to an opening 748 of the housing 708 of the aerosol provision device. In some examples, the outer cylinder may be a double-walled vacuum tube constructed of stainless steel so as to maintain heat generated by the resistive heating element(s) within the outer cylinder, and more particularly, maintain heat generated by the resistive heating element(s) within the aerosol-generating material.

Like the electric heater 706, the resistive heating element(s) 744 may have a variety of different configurations, and vary in number from one resistive heating element to a plurality of resistive heating elements. As shown, the resistive heating element(s) may extend from a receiving base 746 of the aerosol provision device 502. In some examples, the resistive heating element(s) may be located at or around an approximate radial center of the heated end 636 of the consumable 504 when inserted into the aerosol provision device. In some examples, the resistive heating element(s) may penetrate into the heated end of the consumable and in direct contact with the aerosol-generating material. In other examples, the resistive heating element(s) may be located inside (but out of direct contact with) a cavity defined by an inner surface of the heated end of the consumable.

In some examples, the resistive heating element(s) 744 of the electric heater 706 may be connected in an electrical circuit that includes the power source 710 such that electric current produced by the power source may pass through the resistive heating element(s). The passage of the electric current through the resistive heating element(s) may in turn cause the resistive heating element(s) to produce heat through resistance (Joule) heating.

In other examples, the electric heater 706 including the outer cylinder 742 and the resistive heating element(s) 744 may be configured to perform induction heating in which the outer cylinder may be connected in an electrical circuit that includes the power source 710, and the resistive heating element(s) may be connected in another electrical circuit. In this configuration, the outer cylinder and resistive heating element(s) may function as a transformer in which the outer cylinder is an induction transmitter, and the resistive heating element(s) is/are an induction receiver. In some of these examples, the outer cylinder and the resistive heating element(s) may parts of the aerosol provision device 502. In other of these examples, the outer cylinder may be a part of the aerosol provision device, and the resistive heating element(s) may be a part of the consumable 504.

The outer cylinder 730 may be provided an alternating current directly from the power source 710, or indirectly from the power source in which an inverter (as part of the circuitry 712) is configured to convert direct current from the power source to an alternating current. The alternating current drives the outer cylinder to generate an oscillating magnetic field, which induces eddy currents in the resistive heating element(s) 744. The eddy currents in turn cause the resistive heating element(s) to generate heat through resistance (Joule) heating. In these examples, the resistive heating element(s) may be wirelessly heated to form an aerosol from the aerosol-generating material 624 positioned in proximity to the resistive heating element(s).

In various example implementations, the aerosol provision device 502 may include an air intake 750 (e.g., one or more openings or apertures) in the housing 708 (and perhaps also the receiving base 746) to enable airflow into the receiving chamber 722. When a user draws on the mouth end 638 of the consumable 504, the airflow may be drawn through the air intake into the receiving chamber, pass into the consumable, and drawn through the aerosol-generating material 624. The airflow may be detected by the sensor 714, and the electric heater 706 may be activated to energize the aerosol-generating material to generate an aerosol. The airflow may combine with the aerosol that is whisked, aspirated or otherwise drawn out an opening at the mouth end of the aerosol provision system. In examples including the filter 640, the airflow combined with the aerosol may be drawn out an opening of the filter at the mouth end.

The disclosure also provides a method for the preparation of an aerosol precursor composition, comprising combining an organic acid component 136 with one or more one or more of each of a number of constituents such as active substance 126, flavorant 128, aerosol-former material 130 or other functional material 132. The components of aerosol precursor composition 124 can be combined in various orders and two or more components can, in some embodiments, be pre-mixed and added to the composition together in pre-mix form. In other embodiments, the components can be independently added to the composition. In one embodiment, at least one of the organic acids of the organic acid component 136 is first combined with nicotine, e.g., in water, and subsequently combined with other components as described in U.S. Patent Application Publication No. 2019/0116863 to Dull et al., which is incorporated herein by reference in its entirety.

In addition, the disclosure provides kits that provide a variety of components as described herein. For example, a kit may comprise a control body with one or more aerosol generating components/cartridges (including at least one such component comprising a liquid aerosol precursor composition as provide herein). In further embodiments, a kit may comprise a plurality of aerosol generating components/cartridges. In the above embodiments, the aerosol generating components or the control bodies may be provided with a heating member inclusive thereto. A kit may further comprise one or more charging components and/or one or more batteries. The kits may further include a case (or other packaging, transporting, or storage component) that accommodates one or more of the further kit components. The case could be a reusable hard or soft container. Further, the case could be simply a box or other packaging structure.

Many modifications and other implementations of the disclosure will come to mind to one skilled in the art to which this disclosure pertains having the benefit of the teachings presented in the foregoing descriptions and the associated figures. Therefore, it is to be understood that the disclosure is not to be limited to the specific implementations disclosed herein and that modifications and other implementations are intended to be included within the scope of the appended claims. Although specific terms are employed herein, they are used in a generic and descriptive sense only and not for purposes of limitation.

### EXPERIMENTALS

### Example 1: Example Formulations and Preparation

Aerosol precursor compositions are prepared according to the following general components and amounts in Table 1. Percentages are reported in weight, based on the total weight of the aerosol precursor composition.

TABLE 1
Example formulations

| | | Organic acids | | | | | |
|---|---|---|---|---|---|---|---|
| | | Lactic | Benzoic | Levulinic | | Aerosol former (propylene | |
| Sample | Nicotine | Acid | Acid | Acid | Flavor | glycol & glycerin) | Water |
| A | 1.5% | 0.17% | 0.17% | 0.13% | 15- | 60-80% | 2-3% |
| | | | | | 25% | | |
| B | 1.5% | 0.24% | 0.24% | 0.54% | 15- | 60-80% | 2-3% |
| | | | | | 25% | | |
| C | 1.5% | 0.42% | 0.56% | 0.64% | 15- | 60-80% | 2-3% |
| | | | | | 25% | | |
| D | 2.4% | 0.27% | 0.27% | 0.21% | 15- | 60-80% | 2-3% |
| | | | | | 25% | | |
| E | 3.5% | 0.39% | 0.40% | 0.30% | 15- | 60%-80% | 2-3% |
| | | | | | 25% | | |
| F | 5% | 0.56% | 0.56% | 0.43% | 15- | 60%-80% | 2-3% |
| | | | | | 25% | | |
| G | 2.4% | 0.39% | 0.38% | 0.86% | 15- | 60-80% | 2-3% |
| | | | | | 25% | | |
| H | 2.4% | 0.67% | 0.90% | 1.0% | 15- | 60-80% | 2-3% |
| | | | | | 25% | | |
| I | 3.5% | 0.49% | 1.3% | 0.63% | 15- | 60%-80% | 2-3% |
| | | | | | 25% | | |
| J | 3.5% | 0.56% | 0.55% | 1.25% | 15- | 60%-80% | 2-3% |
| | | | | | 25% | | |
| K | 3.5% | 0.97% | 1.3% | 1.5% | 15- | 60%-80% | 2-3% |
| | | | | | 25% | | |
| L | 5% | 0.80% | .79% | 1.8% | 15- | 60%-80% | 2-3% |
| | | | | | 25% | | |
| M | 5% | 1.1% | 1.7% | 0.50% | 15- | 60%-80% | 2-3% |
| | | | | | 25% | | |
| N | 5% | 1.4% | 1.9% | 2.1% | 15- | 60%-80% | 2-3% |
| | | | | | 25% | | |

Various aerosol precursor compositions are prepared based on the components and amounts provided in Table 1. The benzoic acid is first combined with aerosol former material; nicotine in additional aerosol former material is added thereto. To this mixture is added lactic acid, levulinic acid, and water. Additional aerosol former material can then be added and the flavorant is generally added to this mixture last. The resulting solution is mixed thoroughly. It is noted that the preparation of such formulations is not limited to this exact order of addition, and the components of Example 1 can be combined in a number of ways without departing from the present disclosure.

### Example 2: Comparative Sensory Panels

General Method

Informal sensory panels were conducted on various example formulations, in particular, to compare formulations containing two organic acids to formulations containing three organic acids, as described herein. Various tri-acid aerosol precursor compositions were prepared via the general methods provided in Example 1, and corresponding di-acid (or mono-acid) aerosol precursor compositions were similarly prepared as comparative formulations. Each formulation was prepared using a comparable base preparation (including propylene glycol and glycerin as aerosol formers and water), with varying amounts of nicotine and organic acids and varying types of flavorants, as described in more detail herein below.

Each aerosol precursor composition was introduced as the e-liquid within an electronic cigarette and participants were asked to vape and rank each e-liquid formulation on a number of factors. Participants were asked to rank each e-liquid on a scale from 0 to 100, with 0 being the very worst and 100 being the very best, with a rating of “indifferent” at 50 (referred to herein below as the “average sensory score”). Participants were then asked to evaluate the overall taste of the e-liquid and the aftertaste on a scale of 1 to 5. Participants were asked to rank each of the following characteristics of each e-liquid on a scale of 1 to 7 (with 1 being “low” and 7 being “high”): amount of smoothness (and participant's ideal amount of smoothness), amount of harshness (and participant's ideal amount of harshness), amount of throat impact (and participant's ideal amount of throat impact), and amount of aftertaste. Participant's rankings were compiled as provided below.

Sensory Study 1:

A first set of formulations containing 3.5% nicotine (with equivalent amounts of propylene glycol, glycerin, and water) and two organic acids (with 0.4 molar equivalents of lactic acid to nicotine and 0.5 molar equivalents of benzoic acid to nicotine) was prepared according to the general method of Example 1 with five different flavorants. A second set of comparable formulations but with slightly greater lactic acid content was similarly prepared (with 0.5 molar equivalents of lactic acid to nicotine and 0.5 molar equivalents of benzoic acid to nicotine) with the same five flavorants. Finally, a third set of comparable formulations but containing three organic acids was prepared (with 0.25, 0.5, and 0.25 molar equivalents of lactic acid, benzoic acid, and levulinic acid, respectively) with the same five flavorants. The results of an informal sensory panel as described above for this set of e-liquids are presented in Table 2.

TABLE 2
Formulations and Average Sensory Scores for Sensory Study 1

| | | Molar Equivs | Average | | |
|---|---|---|---|---|---|
| | | lactic/benzoic/ | Sensory | | |
| Flavor | Nic % | levulinic | Score | STDev | % RSD |
| Flavor 1 | 3.5 | 0.4/0.5/0 | 78 | 10 | 13 |
| Flavor 2 | 3.5 | 0.4/0.5/0 | 78 | 9 | 12 |
| Flavor 3 | 3.5 | 0.4/0.5/0 | 73 | 9 | 13 |
| Flavor 4 | 3.5 | 0.4/0.5/0 | 66 | 19 | 28 |
| Flavor 5 | 3.5 | 0.4/0.5/0 | 73 | 9 | 12 |
| Flavor 1 | 3.5 | 0.5/0.5/0 | 80 | 8 | 10 |
| Flavor 2 | 3.5 | 0.5/0.5/0 | 78 | 8 | 10 |
| Flavor 3 | 3.5 | 0.5/0.5/0 | 76 | 9 | 12 |
| Flavor 4 | 3.5 | 0.5/0.5/0 | 75 | 18 | 24 |
| Flavor 5 | 3.5 | 0.5/0.5/0 | 76 | 15 | 20 |
| Flavor 1 | 3.5 | .25/0.5/.25 | 74 | 18 | 25 |
| Flavor 2 | 3.5 | .25/0.5/.25 | 75 | 13 | 18 |
| Flavor 3 | 3.5 | .25/0.5/.25 | 67 | 24 | 36 |
| Flavor 4 | 3.5 | .25/0.5/.25 | 76 | 11 | 15 |
| Flavor 5 | 3.5 | .25/0.5/.25 | 74 | 18 | 25 |

As shown in Table 2, for both Flavor 4 and Flavor 5, addition of levulinic acid and reduction in lactic acid (i.e., the tri-acid formulations) improved the average sensory score in comparison to at least one of the tested e-liquids with no levulinic acid.

This data of Table 2 also illustrates certain benefits associated with the inclusion of higher amounts of lactic acid in various embodiments of di-acid formulations. Specifically, as shown, for the majority of flavors, the formulations comprising 0.5/0.5 lactic acid/benzoic acid scored better with respect to average sensory score than corresponding formulations comprising less lactic acid (0.4/0.5). This data suggests that increasing the lactic acid is advantageous in the dual-acid formulations, particularly where the molar equivalents of lactic acid are at least as high as the molar equivalents of benzoic acid.

Sensory Study 2:

A first set of formulations, all containing 5% nicotine (with equivalent amounts of propylene glycol, glycerin, and water) and two organic acids (with 0.44 molar equivalents of lactic acid to nicotine and 0.46 molar equivalents of benzoic acid to nicotine) was prepared according to the general method of Example 1 with five different flavorants. A second set of comparable formulations but containing three organic acids was prepared (with 0.40, 0.46, and 0.14 molar equivalents of lactic acid, benzoic acid, and levulinic acid, respectively) with the same five flavorants. Finally, a comparable formulation but containing 0.29, 0.21, and 0.50 molar equivalents of lactic acid, benzoic acid, and levulinic acid, respectively was prepared with one of the fiver flavorants. The results of an informal sensory panel as described above for this set of e-liquids are presented in Table 3.

TABLE 3
Formulations and Average Sensory Scores for Sensory Study 2

| | | Molar Equivs | Average | | |
|---|---|---|---|---|---|
| | | lactic/benzoic/ | Sensory | | |
| Flavor | Nic % | levulinic | Score | STDev | % RSD |
| Flavor 3 | 5 | .29/.21/.50 | 80 | 9 | 11 |
| Flavor 1 | 5 | .44/.46/0 | 71 | 18 | 25 |
| Flavor 2 | 5 | .44/.46/0 | 75 | 10 | 14 |
| Flavor 3 | 5 | .44/.46/0 | 72 | 13 | 19 |
| Flavor 4 | 5 | .44/.46/0 | 66 | 16 | 24 |
| Flavor 5 | 5 | .44/.46/0 | 76 | 12 | 16 |
| Flavor 1 | 5 | .40/.46/.14 | 67 | 17 | 25 |
| Flavor 2 | 5 | .40/.46/.14 | 74 | 13 | 17 |
| Flavor 3 | 5 | .40/.46/.14 | 76 | 17 | 22 |
| Flavor 4 | 5 | .40/.46/.14 | 69 | 16 | 24 |
| Flavor 5 | 5 | .40/.46/.14 | 69 | 15 | 22 |

As shown in the top entry of Table 3, the e-liquid with the lowest lactic acid and benzoic amounts and the highest levulinic acid amount achieved a superior average sensory score as compared to all other tested e-liquids, regardless of flavor. Adding only a small amount of levulinic acid without significant reduction in lactic acid and benzoic acid amounts generally did not improve the sensory score for most flavors. This top entry was noted by participants to be ideal, based on essentially the same rankings for ideal and perceived traits of smoothness, harshness, throat impact, and aftertaste.

Sensory Study 3:

A set of formulation containing various amounts of nicotine with a single flavor were prepared according to the method outlined above in Example 1 with different ratios of three two organic acids (lactic acid, benzoic acid, and levulinic acid). The results of an informal sensory panel as described above for this set of e-liquids are presented in Table 4.

TABLE 4
Formulations and Average Sensory Scores for Sensory Study 3

| | | Molar Equivs | Average | | |
|---|---|---|---|---|---|
| | | lactic/benzoic/ | Sensory | | |
| Flavor | Nic % | levulinic | Score | STDev | % RSD |
| Flavor 1 | 5 | .26/.46/.18 | 76 | 14 | 18 |
| Flavor 1 | 5 | .40/.46/0 | 75 | 16 | 21 |
| Flavor 1 | 3.5 | .26/.46/.18 | 79 | 12 | 15 |
| Flavor 1 | 2.4 | .26/.46/.18 | 77 | 13 | 17 |
| Flavor 1 | 1.5 | .26/.46/.18 | 75 | 16 | 21 |
| Flavor 1 | 5.0 | .29/.21/.50 | 75 | 14 | 19 |
| Flavor 1 | 3.5 | .29/.21/.50 | 76 | 12 | 16 |
| Flavor 1 | 2.4 | .29/.21/.50 | 79 | 13 | 17 |
| Flavor 1 | 1.5 | .29/.21/.50 | 73 | 19 | 25 |

As shown by the data of Table 4, as the nicotine level was decreased, reducing the benzoic acid amount and raising the levulinic acid amount generally improved the overall sensory score for the e-liquids.

Sensory Study 4:

Two formulations containing 5% nicotine with different berry flavorants were prepared according to the method outlined above in Example 1 with the same molar ratios of three organic acids (lactic acid, benzoic acid, and levulinic acid). These formulations were compared against a comparable formulation with a commercial berry flavorant prepared with lactic acid as the only organic acid. The results of an informal sensory panel as described above for this set of e-liquids are presented in Table 5.

TABLE 5
Formulations and Average Sensory Scores for Sensory Study 4

| | | Molar Equivs | Average | | |
|---|---|---|---|---|---|
| | | lactic/benzoic/ | Sensory | | |
| Flavor | Nic % | levulinic | Score | STDev | % RSD |
| Berry | 5 | .26/.46/.18 | 79 | 13 | 17 |
| Flavor 1 | | | | | |
| Berry | 5 | .26/.46/.18 | 77 | 14 | 18 |
| Flavor 2 | | | | | |
| Commercial | 5 | 1/0/0 | 69 | 13 | 20 |
| Berry | | | | | |
| Flavor | | | | | |

As shown by the data of Table 5, the tri-acid formulations (the first and second entries) scored significantly better in average sensory score than the formulation comprising only lactic acid.

What is claimed is:

1. A liquid aerosol precursor composition adapted for use in an aerosol delivery device, comprising: at least one aerosol former, nicotine, benzoic acid, lactic acid, and levulinic acid, wherein benzoic acid is present in a molar ratio of benzoic acid to nicotine of at least 0.15, wherein lactic acid is present in a molar ratio of lactic acid to nicotine of at least 0.2, and wherein levulinic acid is present in a molar ratio of levulinic acid to nicotine of at least 0.12.

2. The liquid aerosol precursor composition of claim 1, wherein the molar ratio of benzoic acid to nicotine is no more than 0.5.

3. The liquid aerosol precursor composition of claim 1, wherein the molar ratio of lactic acid to nicotine is no more than 0.5.

4. The liquid aerosol precursor composition of claim 1, wherein the molar ratio of levulinic acid to nicotine is no more than 0.6.

5. The liquid aerosol precursor composition of claim 1, wherein the molar ratio of benzoic acid to nicotine is 0.18 to 0.5.

6. The liquid aerosol precursor composition of claim 1, wherein the molar ratio of lactic acid to nicotine is 0.23 to 0.5.

7. The liquid aerosol precursor composition of claim 1, wherein the molar ratio of levulinic acid to nicotine is 0.15 to 0.55.

8. The liquid aerosol precursor composition of claim 1, comprising less than 0.3 molar equivalents of lactic acid to nicotine and less than 0.3 molar equivalents of benzoic acid to nicotine.

9. The liquid aerosol precursor composition of claim 1, wherein the molar ratio of levulinic acid to nicotine is higher than the molar ratio of lactic acid to nicotine and/or higher than the molar ratio of benzoic acid to nicotine.

10. The liquid aerosol precursor composition of claim 1, wherein the molar ratio of levulinic acid to nicotine is higher than the molar ratio of lactic acid to nicotine and higher than the molar ratio of benzoic acid to nicotine.

11. The liquid aerosol precursor composition of claim 1, wherein the combined molar ratio of acids to nicotine is at least 0.7.

12. The liquid aerosol precursor composition of claim 1, wherein nicotine is present in an amount of about 0.5 to about 10% by weight, based on the total weight of the liquid aerosol precursor composition.

13. The liquid aerosol precursor composition of claim 1, wherein the aerosol former comprises at least one polyhydric alcohol.

14. The liquid aerosol precursor composition of claim 13, wherein the at least one polyhydric alcohol is selected from the group consisting of glycerin, propylene glycol, and mixtures thereof.

15. The liquid aerosol precursor composition of claim 13, wherein the aerosol former comprises one or more polyhydric alcohols, and where the one or more polyhydric alcohols are present in an amount of about 50% by weight or higher, based on the total weight of the liquid aerosol precursor composition.

16. The liquid aerosol precursor composition of claim 15, wherein the aerosol former comprises glycerin and propylene glycol, and wherein the glycerin is present in an amount of about 40% by weight or higher and the propylene glycol is present in an amount of about 5% by weight or higher, based on the total weight of the liquid aerosol precursor composition.

17. The liquid aerosol precursor composition of claim 16, wherein the glycerin is present in an amount of about 45% to about 70% by weight and the propylene glycol is present in an amount of about 6% to about 40% by weight, based on the total weight of the liquid aerosol precursor composition.

18. The liquid aerosol precursor composition of claim 1, comprising no more than about 5% by weight of water, based on the total weight of the liquid aerosol precursor composition.

19. The liquid aerosol precursor composition of claim 18, comprising no more than about 3% by weight of water, based on the total weight of the liquid aerosol precursor composition.

20. The liquid aerosol precursor composition of claim 1, wherein the liquid aerosol precursor composition is substantially free or completely free of one or more of phosphoric acid, acetic acid, and pyruvic acid.

21. The liquid aerosol precursor composition of claim 1, further comprising one or more flavorants in a total amount of about 30% by weight or less, based on the total weight of the liquid aerosol precursor composition.

22. The liquid aerosol precursor composition of claim 1, wherein the pH of the liquid aerosol precursor composition is about 5 to about 7.5.

23. An aerosol delivery device, comprising:
a housing enclosing a chamber containing the liquid aerosol precursor composition of claim 1,
a heat source in fluid communication with the chamber and configured to heat the liquid aerosol precursor composition to form an aerosol; and
an aerosol pathway positioned to carry the aerosol to a mouth-end of the aerosol delivery device.

24. The aerosol delivery device of claim 23, wherein the heat source comprises an electrically powered heating element, and the aerosol delivery device further comprises a power source electronically connected to the heating element.

25. The aerosol delivery device of claim 24, further comprising a controller configured to control the power transmitted by the power source to the heating element.

26. A kit, comprising:
a control body; and
one or more cartridges, each cartridge comprising a housing enclosing a chamber containing the liquid aerosol precursor composition of claim 1.

27. The kit of claim 26, further comprising one or more charging components or one or more batteries.
